(19) United States
(12) Patent Application Publication
(10) Pub. No.: US 2017/0202459 A1
(43) Pub. Date: Jul. 20, 2017

(54) WIRELESS MONITORING SYSTEM

(71) Applicant: Zansors LLC, Tysons, VA (US)

(72) Inventor: Hung CAO, Kenmore, WA (US)

(73) Assignee: Zansors LLC, Tysons, VA (US)

(21) Appl. No.: 15/410,120

(22) Filed: Jan. 19, 2017

Related U.S. Application Data
(60) Provisional application No. 62/280,306, filed on Jan. 19, 2016.
(60) Provisional application No. 62/316,105, filed on Mar. 31, 2016.

(51) Int. Cl. A61B 5/00; A61B 5/0404; A61B 5/04; A61B 5/0205; A61B 5/021

(52) U.S. Cl. CPC A61B 5/0006; A61B 5/0013; A61B 5/0205; A61B 5/02125; A61B 5/04017; A61B 5/0404; A61B 5/6831; A61B 5/742; A61B 5/0022; A61B 5/6898; A61B 5/02416

(57) ABSTRACT

At least one sensor in a wearable device worn by a user may be configured to detect at least one of an electrocardiogram (ECG) signal and arterial pulse (or photoplethysmogram (PPG)) signal. At least one processing circuit may process the ECG signal, the pulse/PPG signal, or both to determine at least one of a pulse transit time (PTT) and pulse arrival time (PAT). The at least one processing circuit may determine a blood pressure (BP) of the user from the PTT, the PAT, or both.

### CROSS-REFERENCE TO RELATED APPLICATION

This application claims priority from U.S. Provisional Application No. 62/280,306, filed Jan. 19, 2016, the entirety of which is incorporated by reference herein.

### BACKGROUND

Cardiovascular disease (CVD) is a leading of morbidity and mortality. Uncontrolled high blood pressure (BP), or hypertension (HTN), is a common condition leading to serious CVD complications. The increased systemic arterial pressure causes increased afterload on the heart leading to permanent cardiac damage as well as other arteries that can lead to stress on the heart, also known as hypertensive heart disease. About 67 Million American adults (31%) are affected by HTN, 47% of whom maintain normal BP control. Further, HTN has been also found associated with other health issues in particular groups of populations, such as elderly people and pregnant women, to name a few. For instance, HTN disorders during pregnancy accounted for approximately 17% of maternal mortality in the U.S., while stroke and other end-organ dysfunction in the elderly are known to be caused by HTN. While numerous research efforts have been achieved in the last few decades in order to provide therapeutic solutions, regular examinations and check-ups play a key role in prevention and for early treatment. Nevertheless, it is usually troublesome and time consuming to visit a hospital and arrange appointments with doctors to obtain physiological information.

### BRIEF DESCRIPTION OF THE DRAWINGS

FIG. 1 shows a time delay in signals detected at two different sites on the body according to an embodiment of the invention.

FIG. 2 shows an electrical signal and a pressure signal from a heart according to an embodiment of the invention.

FIG. 3 is a series of graphs illustrating relationships between blood pressure and pulse transit time according to an embodiment of the invention.

FIG. 4 is a PTT PIR calculation according to an embodiment of the invention.

FIG. 5A is a wearable device according to an embodiment of the invention.

FIG. 5B is a sensor according to an embodiment of the invention.

FIG. 5C is a flexible sensor unit according to an embodiment of the invention.

FIG. 6 is a sensor according to an embodiment of the invention.

FIG. 7A shows alternate wearable devices according to embodiments of the invention.

FIG. 7B is a sensed signal according to an embodiment of the invention.

FIG. 7C is a sensed signal according to an embodiment of the invention.

FIG. 8 is a circuit according to an embodiment of the invention.

FIG. 9 is a network according to an embodiment of the invention.

FIG. 10 is a data capture process according to an embodiment of the invention.

FIG. 11 is a data analysis process according to an embodiment of the invention.

FIG. 12 is a data collection process according to an embodiment of the invention.

### DETAILED DESCRIPTION OF SEVERAL EMBODIMENTS

Embodiments disclosed herein provide a system and method for wirelessly monitoring a patient's health, such as e.g., the patient's cardiovascular health.

Embodiments disclosed herein provide an armband (referred to as a Z-band) or other wearable device offering continuous monitoring of electrocardiogram (ECG), photoplethysmogram (PPG), and/or BP for home uses. In some embodiments, data may be displayed by an application running e.g., on a smartphone or other mobile device. The armband may transmit data to the device using wireless communications such as e.g., Bluetooth. The application may use algorithms to analyze the data in real-time. The application may include the function to send the data to a registered doctor for online or offline analyses.

Some embodiments may provide hassle-free wearable devices which can serve as e-doctors to monitor patients or even healthy people 24 hours a day, 7 days a week while retaining daily activities. These embodiments may provide an affordable, continuous home BP-monitoring cuffless device that may significantly cut down time and cost in HTN-related healthcare and provide not only a paradigm shift in HTN monitoring, but also in numerous cardiovascular investigations with respect to the effects of different activities, medications, and lifestyle choices on BP.

An example wearable device may include a bioengineered wearable wristband containing a micro-electro-mechanical systems (MEMS)-based sensitive pulse sensor, non-contact electrodes (NCEs) for ECG measurement, and electronic components for data acquisition and communication with a smartphone via Bluetooth. The device may offer unobtrusive 24/7 simultaneous monitoring of ECG and arterial pulse, allowing BP to be analytically calibrated and obtained by optimized algorithms. The wristband may be made of flexible and stretchable polymer that can be worn by any user, thus providing comfort and versatility.

NCE ECG measurements may be obtained without interference from any effects from the skin (sweat, hair, etc.), thus being useful for off-the-clinic measurements. Indeed, the NCE approach for biopotentials acquisition and flexible electronics may allow manufacturing of healthcare devices which can be embedded inside clothing and accessories. The use of a sensitive polymer-based strain sensor for wrist pulse may allow a compact integration for continuous assessment of BP. Further, a cloud computing framework may enable scalable storage and distributed computing environments for the increasing data set sizes. The hardware and device independence provided by the cloud-based machine learning and Internet of Things (IoTs) platforms may support collaborative hypothesis testing and identification of embedded patterns in the data. Accordingly, the disclosed devices may revolutionize the management of patients with HTN as well as provide 24/7 monitoring of BP in general populations, for example to provide public health metrics. The utilization of the ready smartphone ecosystem may help spread the devices to the general public as well as cut down the implementation cost.

Wearable devices constructed and configured as described herein may perform continuous, cuffless BP monitoring while being worn solely on one side of the body and without requiring cross-body contact (e.g., with contact only on one side of the body). For example, such wearable devices may be worn only on an arm or wrist without requiring a separate element on another side of the body and without requiring a wearer to touch an open electrode on the wearable device. Furthermore, a wrist-worn embodiment may be unnoticeably integrated into a watch or bracelet. Accordingly, the devices described herein may integrate the valuable feature of continuous BP monitoring into wearables in the coming era of mobile health (m-Health) and IoTs.

### Blood Pressure Measurement

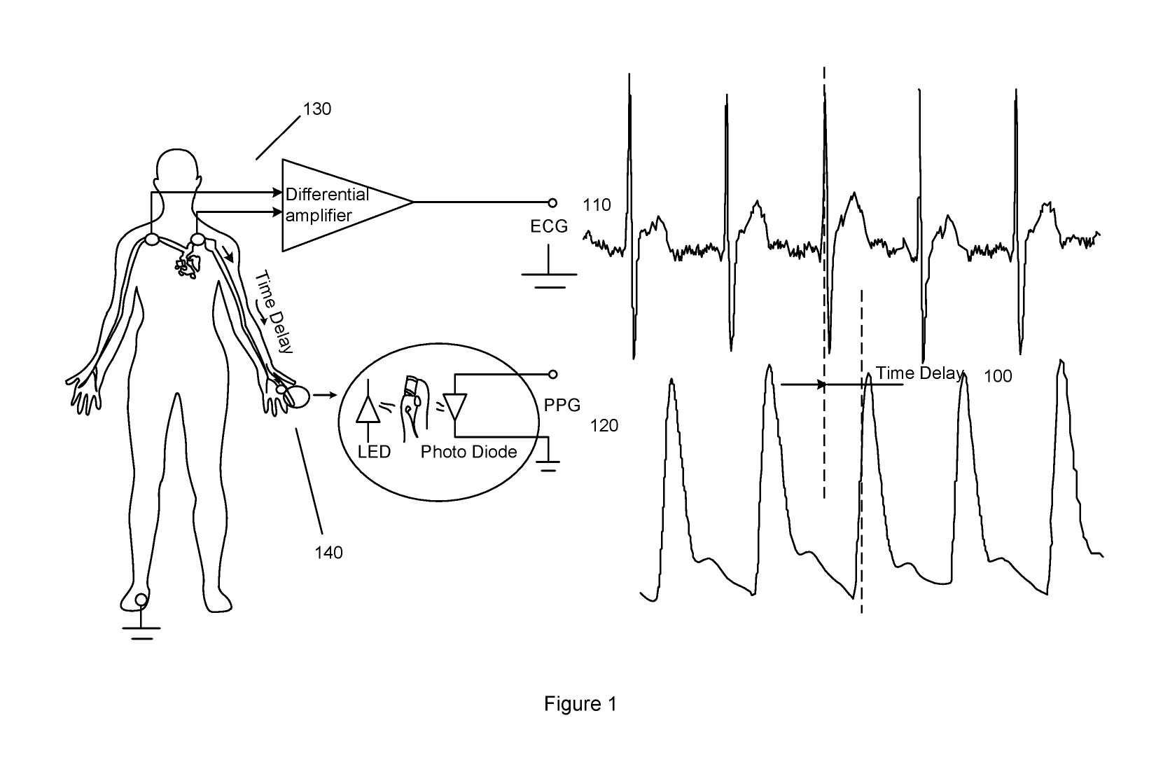

Intravascular BP may be related with the traveling time of blood from the heart to a peripheral organ, which could be measured in the form of pulse transit time (PTT) or pulse arrival time (PAT), for example. FIG. 1 illustrates a time delay 100 in signals 110, 120 detected at two different sites 130, 140 on the body, respectively. Accordingly, wearable devices described herein may use the physical relationship between blood pressure and the traveling/delay time of the cardiac signals in a wearer's cardiovascular system to derive BP for the wearer. Various algorithms and/or mathematical models may be applied to optimize the regression process and calibration of the traveling/delay time and BP.

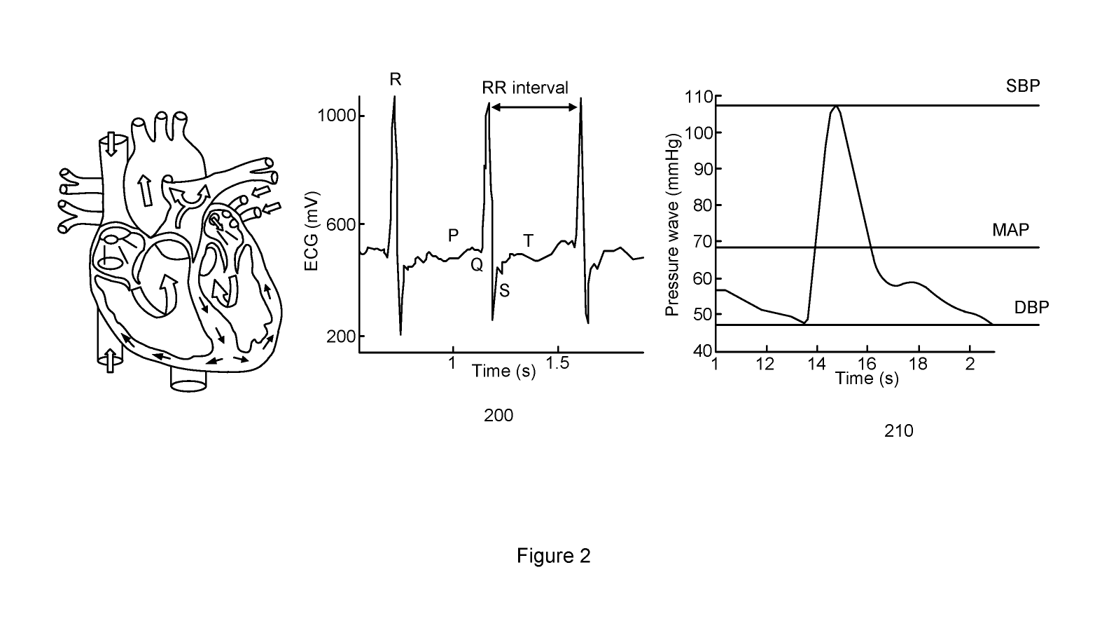

FIG. 2 shows an electrical signal 200 and a pressure signal 210 from a heart. The heart's electrical system, also known as the cardiac conduction system, includes three main parts; sinoatrial (SA) node, atrioventricular (AV) node, and His-Purkinje system. The heart's electrical system recorded as an ECG 200. The SA node, located in the upper portion of the right atrium, is the heart's natural pacemaker. The SA node initiates an electrical signal, indicated as a P-wave on a patient's ECG, and regulates the pace or rate of the heart. Generated electrical signals travel to the right and left atria causing the two upper chambers to contract. The contractions push blood through the valves into both ventricles. The electrical impulse then activates AV nodes situated above the ventricles. The AV nodes, acting as relay stations, prevent immediate travel of the electrical pulse, facilitating right and left atria to empty their blood contents into the two ventricles. The PR interval in the ECG indicates this delay. On release, the electrical signal moves along a pathway around the walls of the heart's ventricles (termed bundle of His), eventually later dividing into Purkinje fibers connected to cells in the walls of left and right ventricles, causing the electrically stimulated ventricles to contract and pump oxygenated blood into the arteries. This entire phase represents the QRS complex of an ECG. Later stages of the ECG represent the recovery phase of the ventricles from the electrical stimulation.

The electro-mechanical function of the heart results in blood ejection into the arterial tree, affecting the blood velocity and generating a systemic pressure wave travelling from the central to peripheral arteries. The pressure wave causes dilation of the arterial walls on its path and moves faster than blood flow. The regular pulse beat felt at the peripheral arteries indicates this pressure wave and not the blood flow. The pressure wave varies periodically between two extreme points. The maximum is Systolic BP (SBP), pressure in the artery due to ventricular contraction, and the minimum is Diastolic BP (DBP), pressure in the artery during each beat. The mean value of the pressure wave is called MAP 210 and may be estimated using equation (1).

$$MAP = DBP + \frac{1}{3} ( SBP - DBP ) \qquad ( 1 )$$

This pressure waveform can be directly obtained using a pulse sensor on the peripheral arteries or indirectly measured through PPG. Since the pressure wave causes the blood volume to change at the peripheral site, it can be detected by measuring the variation of the oxygen content of the blood caused by the influx of oxygenated blood on arrival of the pressure wave, indicated as the first peak on the PPG waveform. Many other vital parameters can be estimated using PPG as discussed below.

The central arteries push blood to narrow distal arteries by expanding during systole and contracting during diastole. This expansion and contraction results in a change of the elastic modulus E of the blood vessels and can be related to the fluid pressure P by equation (2).

E=Eₒe^{αP} (2)

Here, α is vessel's parameter (Euler number) and Eₒ is the Young's modulus for zero arterial pressure. Equation (2) accurately estimates the central arterial pressure if α, Eₒ are updated to account for the age impact on the elasticity due to change in wall composition. Arterial walls are composed of endothelium, elastin, collagen, and smooth muscle (SM) in varying quantities at central and peripheral sites. Different composition as well as gradual replacement of elastin with collagen changes elasticity of the arteries, causing changes in central and peripheral BP. Central arterial elasticity is determined by BP. However, peripheral elasticity is affected by both BP and SM contraction and hence cannot be accurately predicted by (2).

Elasticity of the arteries, in turn, determines the propagation speed of the pressure wave PWV, and a relationship can be obtained between the two using arterial wave propagation models. The model assumes the artery to be an elastic tube with thickness t, diameter d, and blood density ρ, to obtain equation (3) also called the Moens-Kortweg equation.

$$PWV = \sqrt{\frac{tE}{ρ d}} \qquad ( 3 )$$

Combining equations (2) and (3), we obtain Bramwell-Hills and Moens-Kortweg's equation (4), defining a relationship between P and pulse wave velocity (PWV) and hence pulse transit time (PTT) for an artery of length L.

$$PWV = \frac{L}{PTT} = \sqrt{\frac{{tE}_{o} {e}^{α P}}{ρ d}} \qquad ( 4 )$$

Equation (4) indicates that the rise in pressure, with other parameters constant, results in an increase in PWV and inversely effects PTT. The assumption of a tubular arterial system works effectively for central arteries but fails for the tapering peripheral branches. Impedance mismatch due to arterial terminations results in wave reflection, distorting the peripheral pressure waves. Different waveform shapes and pulse pressures result between two different points for the same mean pressure as magnitude and phase of the frequency components of the pressure pulse change. When a pressure wave reaches the end of a vascular tube, a portion of the wave is reflected back towards the heart with reflection coefficient (Γ) given by equation (5) for an arterial segment of terminal impedance Z_{T} and each harmonic component (ω). The net pressure waveform at the site is a sum of incident and this reflected wave.

$$Γ ( ω ) = \frac{{Z}_{T} ( ω ) - {Z}_{0} ( ω )}{{Z}_{T} ( ω ) + {Z}_{0} ( ω )} \qquad ( 5 )$$

Since there is a negligible time delay between the forward and backward waves, superimposition of the two pressure pulse results in an increase in the peripheral pulse pressure. On the other hand, a phase difference of 2*PTT separates the incident and reflected waveform at central arteries, keeping the distortion insignificant. A second smaller peak on a central PPG is indicative of this reflected waveform and is independent of the incident peak. Waveforms having significant wave reflection do not represent correct PTT and should be taken in consideration.

In view of the above, wearable devices and/or other devices with which they communicate can measure BP continuously. In one example, BP may be derived using PTT obtained from ECG and PPG measurements. Using PPT, we can calculate the pulse wave velocity (PWV) as

$$PWV = \frac{d}{PTT} \qquad ( 6 )$$

where d is the distance from heart to the Z-band and can be estimated as 25% of the height of the individual. The BP can be calculated using the following equation:

BP=a×PWV²+b (7)

where a and b are constants to be determined by training an algorithm.

In another example, a relationship between Systolic BP (SBP) and PTT may be modeled based on physical behavior and regression. These mathematical models may have subject-specific constants, A and B, that account for other physical parameters that influence BP, like arterial wall elasticity, age, and blood density. The equation for each subject using different models may be found by linearly curve fitting the measured BPs and the experimentally obtained PTTs. These equations may be subject to change with modifications of the parameters affecting A and B, thus requiring recalibration. One model (8) is based on the inverse relationship between PTT and BP. This model assumes that, at a fixed distance from the heart with other parameters constant, BP depends only on the time that the pulse takes to travel along the distance.

SBP=A/PTT+B (8)

A second model (9) is derived similarly to (8) and relates Systolic BP (SBP) inversely to the square of PTT [8].

SBP=A/PTT² +B (9)

A third model (10) explores the relationship between arterial elasticity and pulse wave velocity (PWV). PWV of the pressure wave is inversely proportional to PTT and depends on the distance between heart and point of measurement. Arterial elasticity is exponentially related to PWV. On basis of this equation, SBP can be related to PWV.

SBP=A×ln(PTT)+B (10)

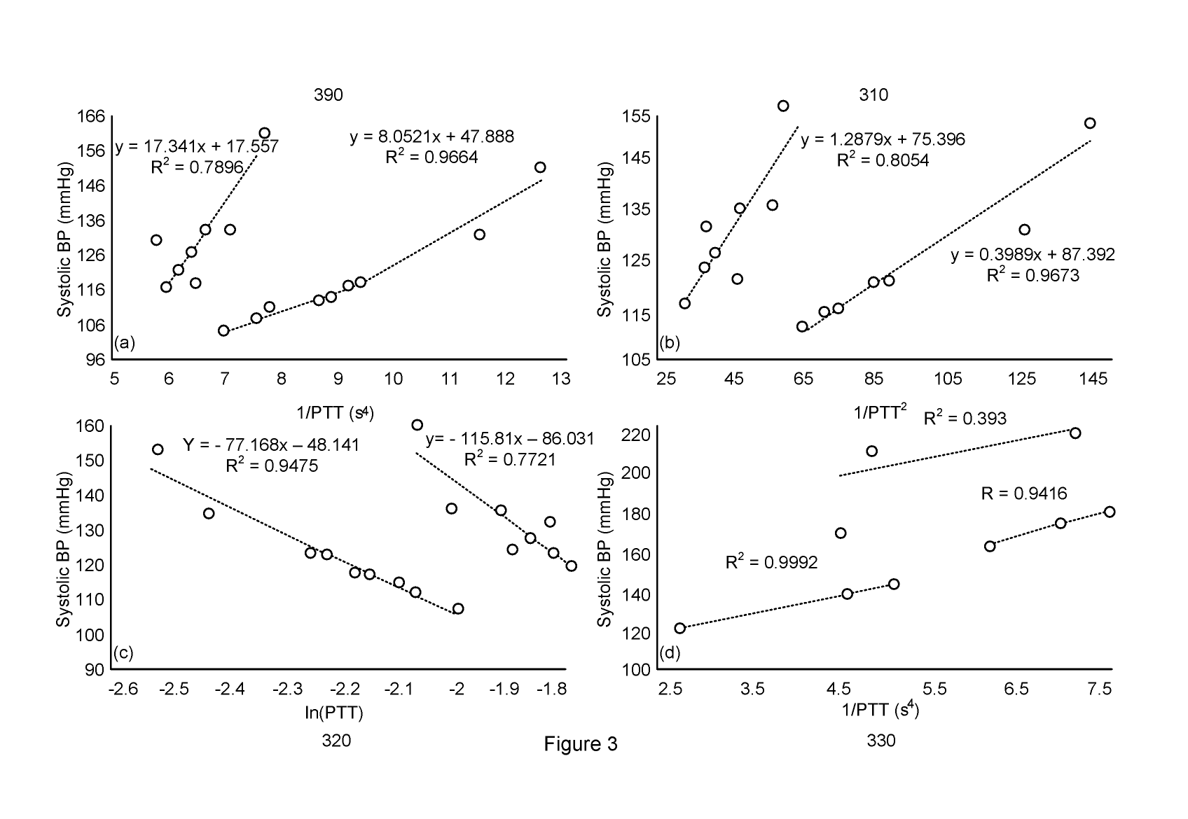

FIG. 3 is a series of graphs illustrating relationships between BP and PTT according to an embodiment of the invention. Specifically, FIG. 3 shows an example relationship between BP and inverse PTT 300, an example relationship between BP and inverse square PTT 310, and an example relationship between BP and ln(PTT) 320. FIG. 3 also shows a graphing of BP of multiple subjects versus inverse PTT 330.

Elasticity of arteries is also directly proportional to pulse pressure in the artery and hence can be related to PWV.

(PP=SBP−DBP) (11)

PP∝PWV² (12)

PWV is the rate at which a pulse propagates, thus it can be estimated as the ratio of distance travelled (length of the arterial branch) and time taken to travel the distance, PTT. Hence, equation (12) can be re-written as (13) with calibrated PPₒ and PTTₒ.

$$PP = {{PP}_{o} ( \frac{{PTT}_{o}}{PTT} )}^{2} \qquad ( 13 )$$

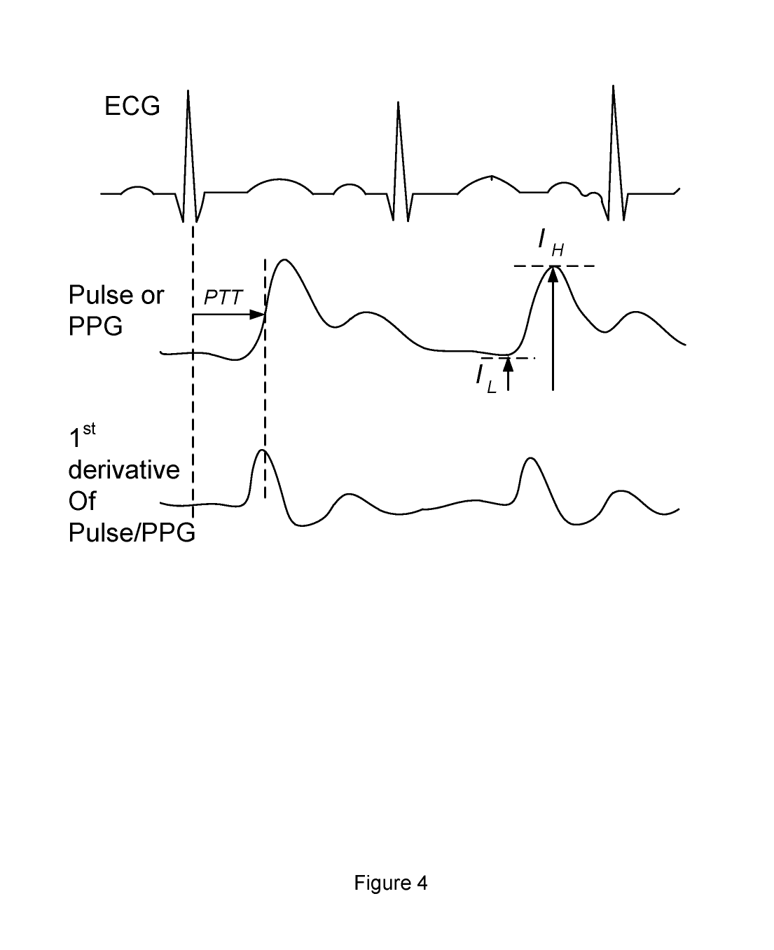

Also, using a two-element Windkessel model of an artery with peripheral resistance (R) and arterial compliance (C), DBP can be estimated as shown in (14).

$$DBP = {P}_{o} {e}^{- \frac{t}{RC}} \qquad ( 14 )$$

where Pₒ is the end-systolic aortic pressure. Since C is a constant for short period, DBP mainly varies with R, which changes with the arterial diameter variation Δd during each pressure wave. This change in diameter can be estimated using PIR calculated as the ratio of peak and foot of pulse/PPG wave of the same cardiac cycle (15), as seen in FIG. 4.

PIR=e^{βΔd} (15)

As Δd is inversely proportional to R, a relationship can be obtained between PIR and DBP as shown in (16), where DBPₒ and PIRₒ is obtained through calibration.

$$DBP = {{DBP}_{o} ( \frac{{PIR}_{o}}{PIR} )}^{2} \qquad ( 16 )$$

SBP, which is the sum of DBPₒ and PPₒ can be estimated as shown in (17).

$$SBP = {{DBP}_{o} ( \frac{{PIR}_{o}}{PIR} )}^{2} + {{PP}_{o} ( \frac{{PTT}_{o}}{PTT} )}^{2} . \qquad ( 17 )$$

The aforementioned mathematical models and algorithms may be used by a wearable device, computer in wireless communication with the wearable device, cloud computing platform, or combination thereof for continuous monitoring of a user's BP.

### Wearable Devices

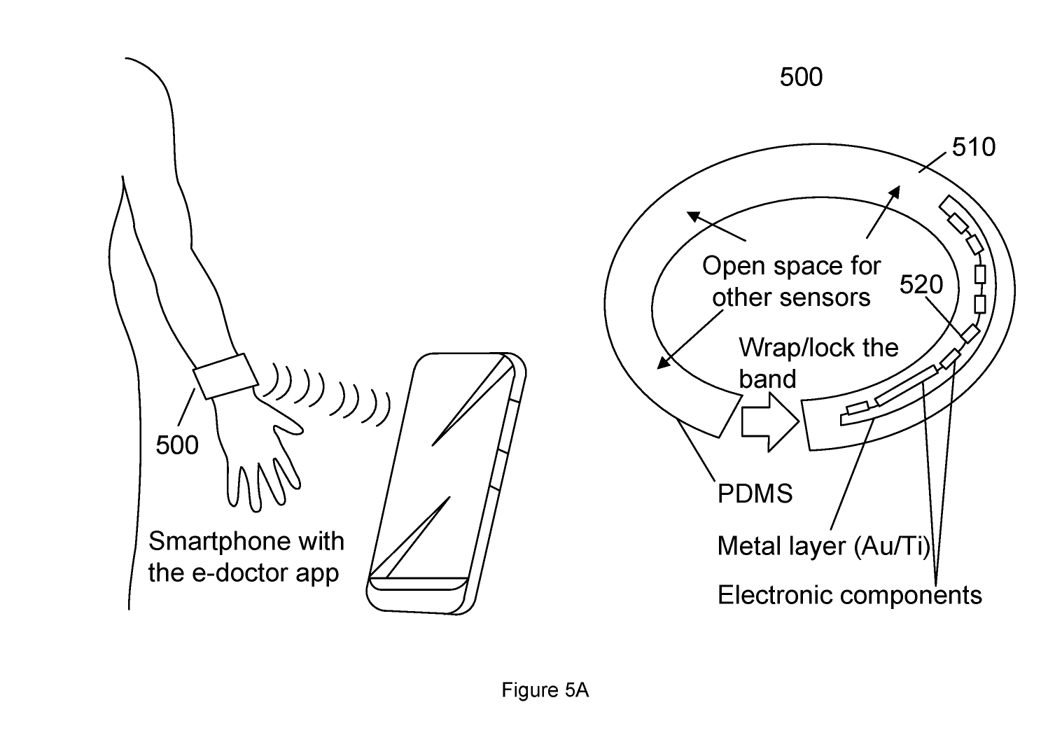

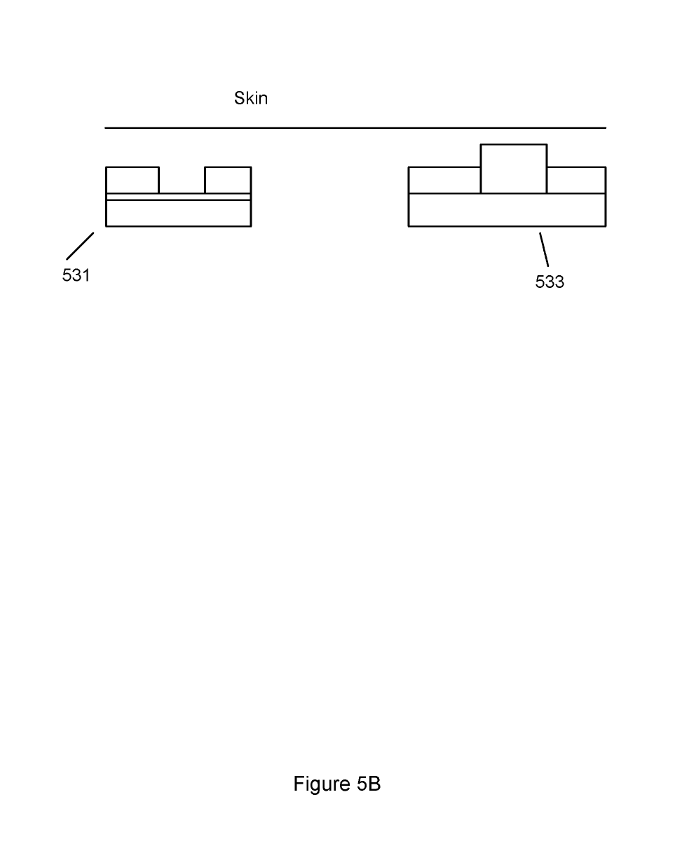

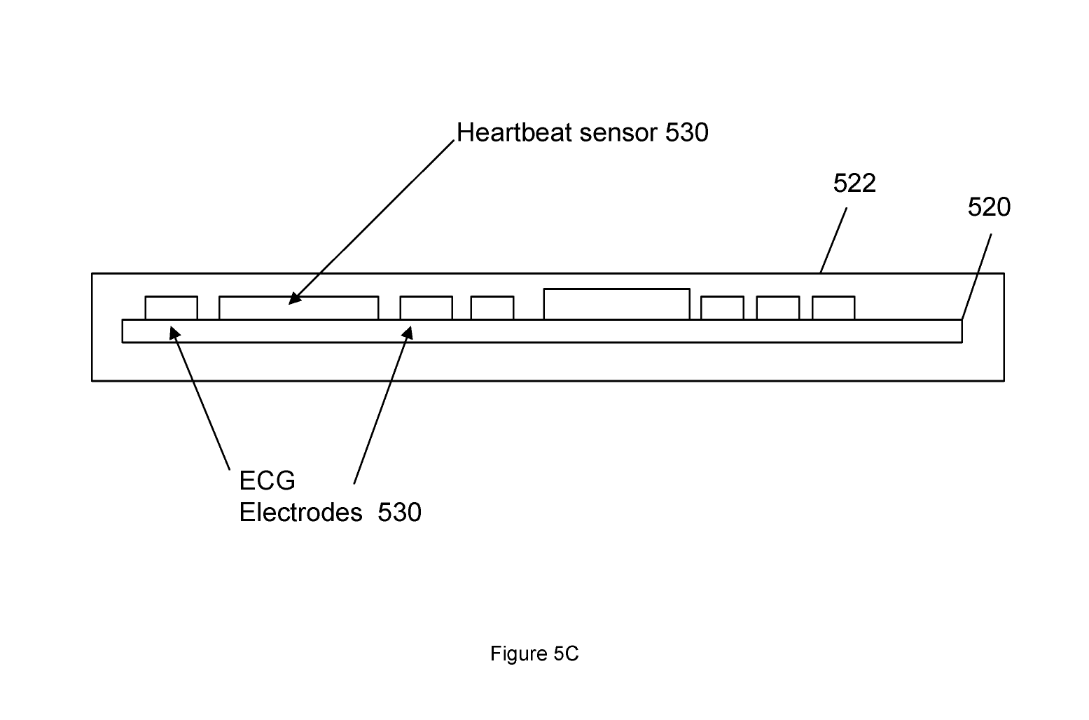

FIG. 5A is a wearable device 500 according to an embodiment of the invention, FIG. 5B shows a detailed view of a sensor 530 of wearable device 500, and FIG. 5C shows a detailed view of a flexible sensor unit 520 of wearable device 500. Wearable device 500 may be configured to measure electrical and/or pressure signals and/or perform calculations as described above in the “Blood Pressure Measurement” section of this document. Specifically, some embodiments of wearable device 500 may include a stretchable arm band containing a multi-electrode array (MEA) with metal routings to mount a PPG chip, a Bluetooth chip, and electronic components to obtain ECG and PPG data and to send the obtained data to a smartphone or other device. The smartphone or other device may process the received data to extract BP information. An algorithm may be used to train the program, and other algorithms may be used to interpret and judge the data.

Wearable device 500 may include flexible band 510 (e.g., polymer band) and flexible sensor unit 520. Flexible sensor unit 520 may include one or more sensors 530. For example, sensors 530 may be formed as part of an MEA. Sensors 530 may include electrodes without 531 and/or with 533 electroplating to reduce the skin-electrode impedance. Flexible sensor unit 520 may include a back-side stacking of two 5-μm parylene C pieces and circuits 522 connected by vias 524, for example.

More specifically, in some embodiments the electrodes and metal routings of flexible sensor unit 520 may be specially designed to be stretchable in order to fit different sizes of users. The MEA may be fabricated with metal lines, and IC chips and discrete components may be integrated therewith. In one example, the metal MEA (3 electrodes) and metal routings may be formed of a double layer of Au on Ti (1 μm/ 0.01 μm) sputtered on a 5-μm parylene C polymer. The metal traces may be covered by a 0.5-μm thick parylene C, while the electrode areas may be exposed. The electrode areas may be thickened by electroplating so that they have a positive height after packaging to guarantee a low impedance of the skin-electrode interface. In some embodiments, non-contact electrodes may be provided instead of or in addition to the contact electrodes. Electrodes of either type may be formed in a variety of sizes and arrangements, e.g., 2×2 mm², 5×5 mm², 1×1 cm², 1×1 cm², etc. Some embodiments may incorporate other sensors such as temperature and humidity sensors as well.

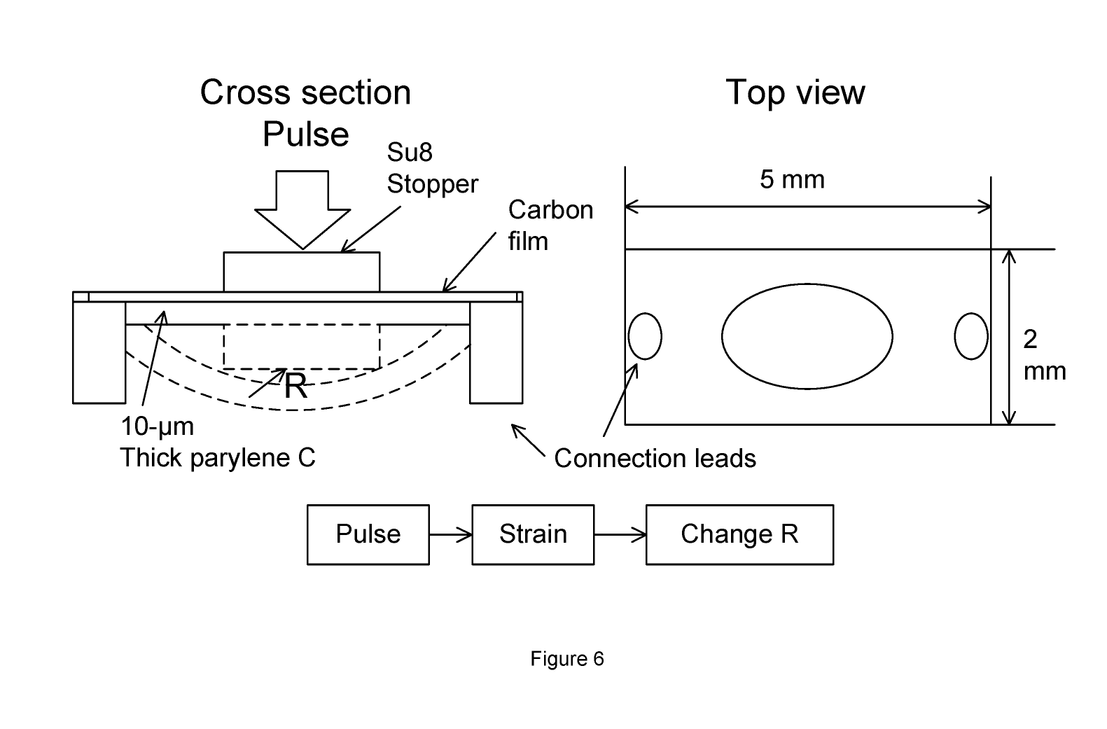

FIG. 6 shows a specific example of a contact sensor 530 according to an embodiment of the invention. Contact sensor 530 may comprise a pulse sensing cell 532 with a stopper 534 (e.g., with a size of 2×5 mm²). The stopper 534 may act as a bump so that the contact force or wrist pulse will be easily detected. The stopper 534 may be made of photoresist SU-8 100 deposited by a conventional photolithography process. For example, the stopper 534 may have a diameter of 1 mm and a thickness of 100 μm. The leads 536 may be connected to the carbon film 538 by conductive epoxy to form thick connection pads so that the pulse sensor 530 can operate.

Flexible sensor unit 520 may include a PPG circuit (e.g., AFE4400 from Texas Instruments) and communication (e.g., Bluetooth or Bluetooth low energy (BLE)) circuit (e.g., RN4020 from Microchip Technology, EZ430-RF2500 from Texas Instruments, or nRF51422 from Nordic Semiconductor). These circuits may be mounted on the polymer with the discrete components on a 5-μm parylene-C flexible circuit. The two pieces of parylene may be attached back-to-back. Vias may be made to connect the electrodes with other components. The assembled circuitry may be packaged in flexible and stretchable silicone to achieve the final flexible band 510. Flexible sensor unit 520 may include a rechargeable battery. Flexible sensor unit 520 may include a low-power embedded microcontroller for capturing and processing sensed signals.

Wearable device 500 may be fabricated by one of a variety of processes. The following is presented as an example fabrication process whereby the MEA and carbon thin-film strain sensor may be fabricated with metal routing lines, ICs, and discrete electronic components.

First, a carbon thin film may be sputtered and patterned by a lift-off process on a 10-μm thick parylene C pre-deposited on a Si wafer. In embodiments using a pulse sensing cell type sensor 530, the sensor(s) 530 may be formed as described above. The NCE, shielding pad, and metal routings may be fabricated on a different parylene membrane. Sputtering may be used to form a double layer of Au on Ti (0.3 μm/0.01 μm). The Au routings may be designed in meandrous form to enhance the stretchability. A Bluetooth low energy (BLE) chip (nRF51422 from Nordic Semiconductor) may be used to transmit NCE ECG and pulse data from the wearable band to a BLE supporting (Bluetooth 4.0 onwards) smartphone and/or computer. The nRF51422 is built with a 32-bit CPU with 256 kB flash and 32 kB RAM. The entire 6×6 mm² package operates with a 2.4 GHz transceiver. The microcontroller has 8 analog channels and a 10-bit analog-to-digital converter (ADC) that will be used to sample the data at 200 Hz. A commercial Li-polymer rechargeable battery may be used. After mounting all components and validating the operation, the entire device may be encapsulated in polydimethylsiloxane (PDMS). The parylene C layer may provide ease of fabrication, while PDMS may add stretchability.

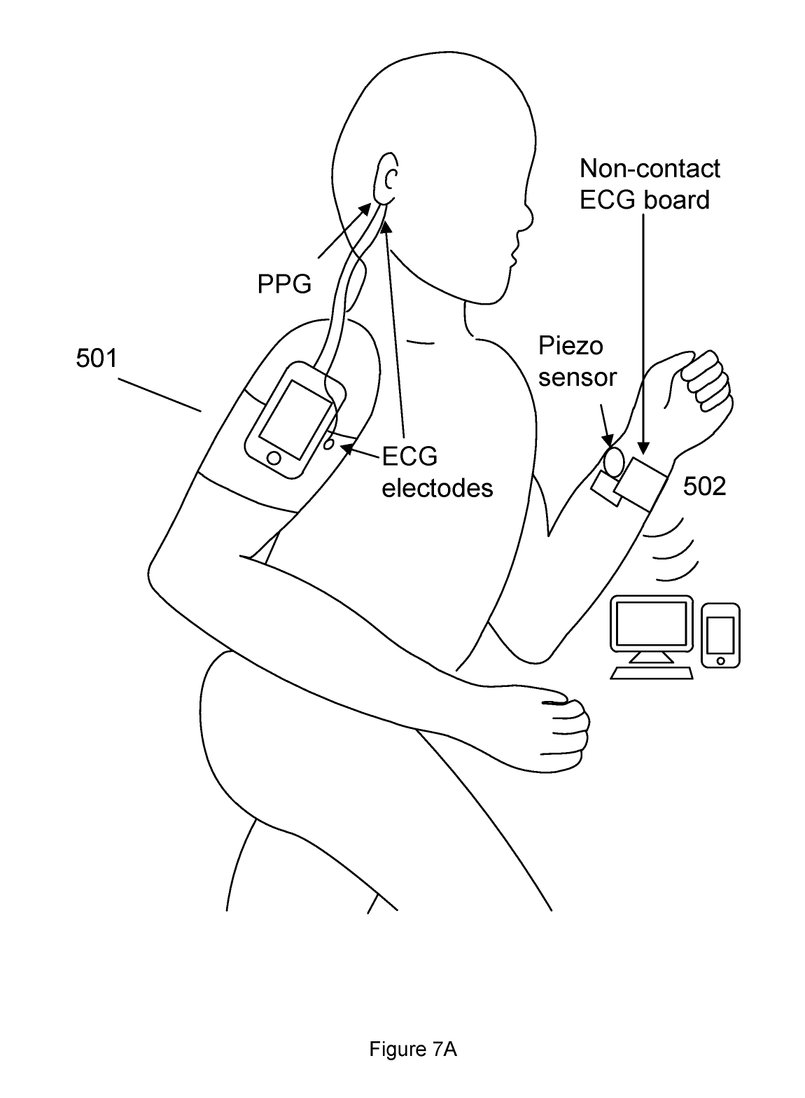

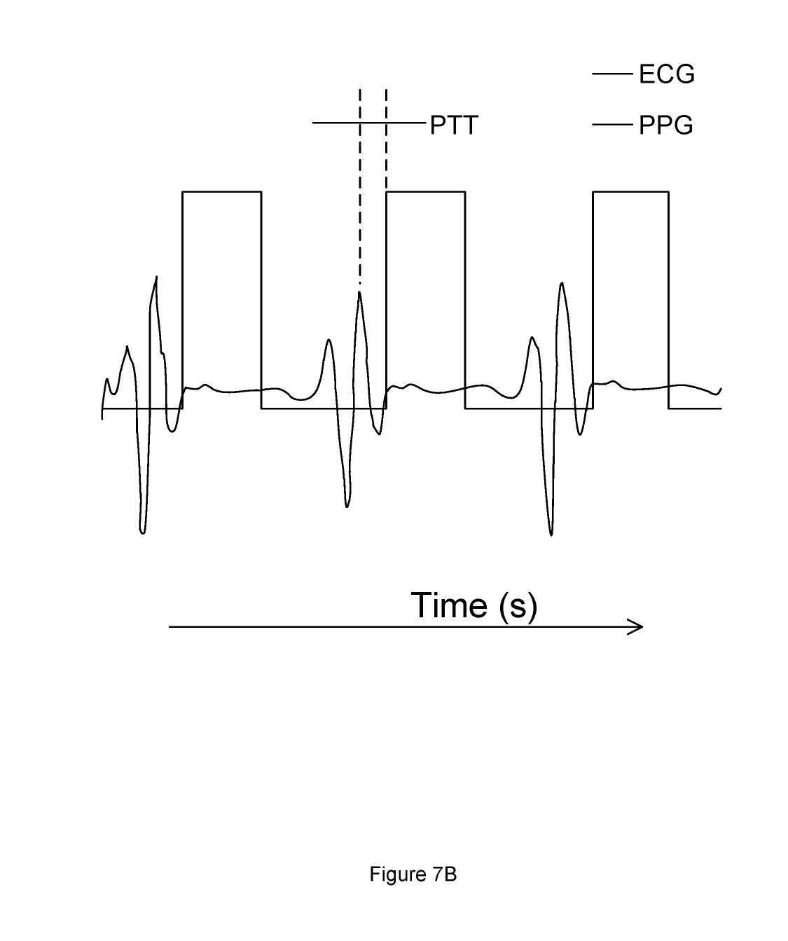

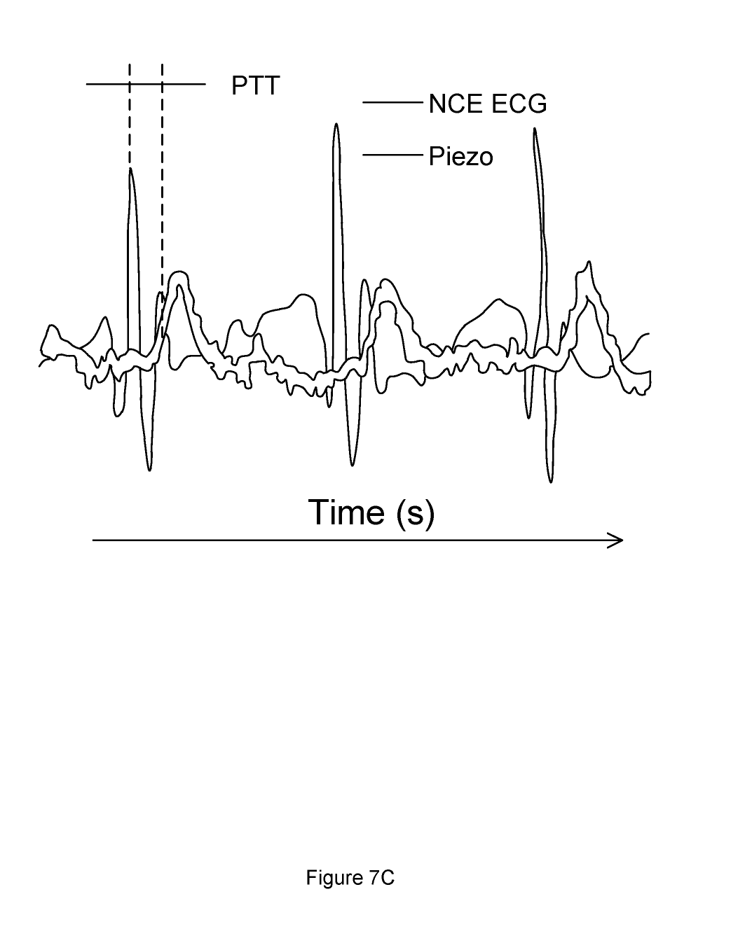

Wearable device 500 may be configured differently depending on how it is meant to be worn. Example embodiments may include a bicep-worn device and a wrist-worn device. FIG. 7A shows a bicep-worn device 501 and a wrist-worn device 502 according to embodiments of the invention.

The bicep-worn device 501 may include a unit for signal filtering and amplification and a unit for wireless data transmission worn on the bicep with wired, contact ECG electrodes attached to the bicep and to the back of the ear, and with a PPG sensor (e.g., from Seeed Studio) clipped to the earlobe. An instrumentation amplifier (e.g., INA331 from Texas Instruments) may be included as a preamplifier. A detected signal may pass through the preamplifier. After the preamplifier stage, the signal may be filtered by a passive high-pass filter followed by two cascaded low-pass filters (e.g., OPA333 from Texas Instruments) in order to obtain the ECG frequency range of 0.5-150 Hz. FIG. 7B shows an example simultaneous recording of ECG and PPG by a bicep-worn device 501.

The wrist-worn device may include a pulse pressure sensor and a non-contact electrode board (NCE) for ECG acquisition. Different from the contact ECG approaches, NCE ECG could be obtained without any effects from the skin (sweat, hair, etc.). The wrist-worn device may include a piezoelectric sensor (e.g., from Audiowell) tailored to fit on the wrist above the radial artery. The NCE may record ECG data by capacitively coupling with the body. The electrode plate may be electrically insulated, therefore acting as a dielectric between the electrode and the subject's skin. An amplifier (e.g., INA116 from Texas Instruments) may be included to amplify the coupling signal. The output may be offset by 1.5 V and passed through a twin-T notch 60 Hz filter with a buffered output. FIG. 7C shows an example simultaneous acquisition of NCE ECG and radial pulse.

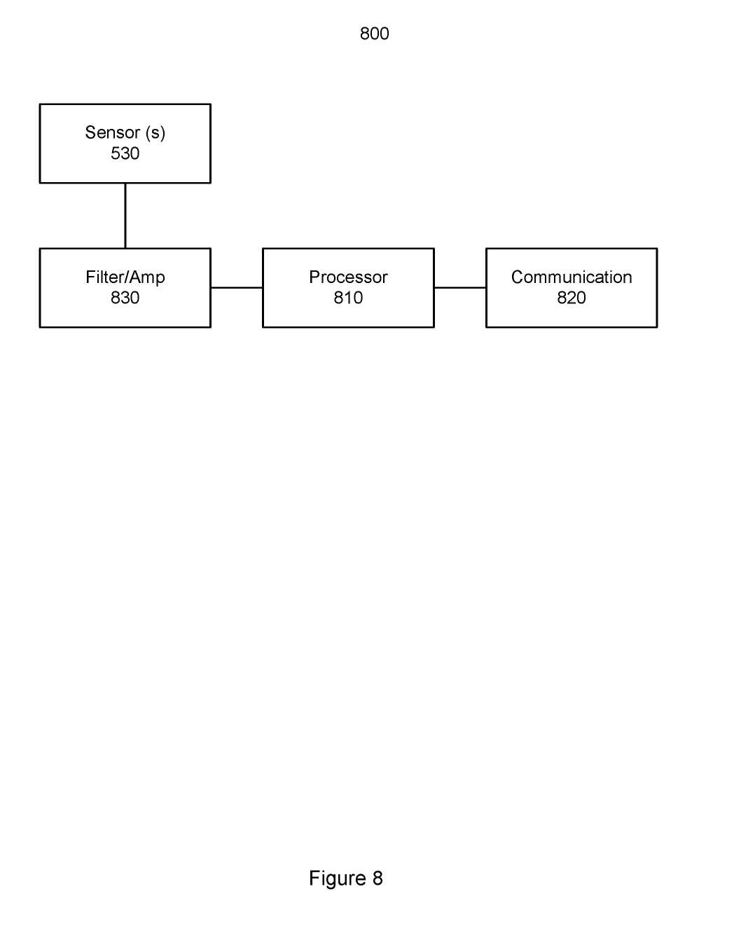

FIG. 8 is a circuit 800 according to an embodiment of the invention. Circuit 800 may be formed on and/or within flexible sensor unit 520 as described above. Circuit 800 may include sensor(s) 530 coupled to filtering and/or amplifying circuitry 830. Sensor(s) 530 may sense user data, and filtering and/or amplifying circuitry 830 may filter and/or amplify the data, as discussed above. Processor 810, coupled to filtering and/or amplifying circuitry 830, may perform pre-processing as discussed above. Communication circuitry 820, coupled to processor 810, may send pre-processed data to a remote device (e.g., using Bluetooth or BTLE) as discussed above.

Processing

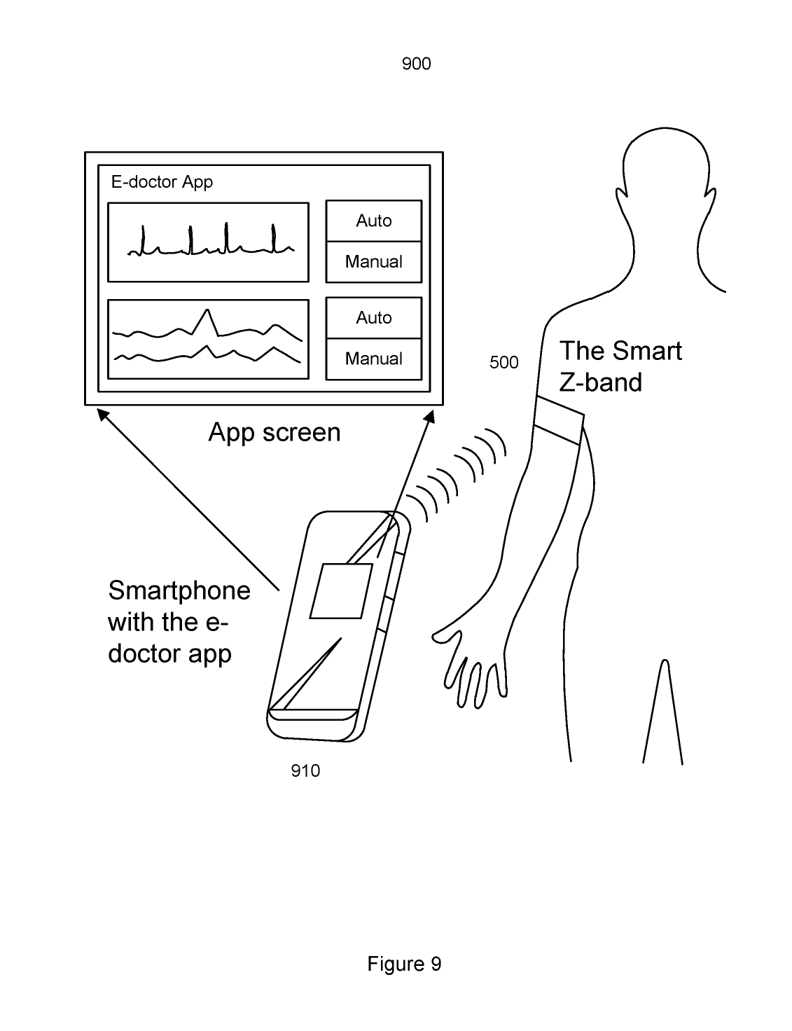

FIG. 9 is a network 900 according to an embodiment of the invention. Network 900 may include wearable device 500 and smartphone or other computer 910 (hereinafter referred to as smartphone 910 for brevity). Smartphone 910 may comprise one or more hardware processors, one or more communication circuits (e.g., configured to communicate with wearable device 500 using Bluetooth or BTLE), display and/or input device(s), memory, and software such as an application that configures smartphone 910 processor to process data gathered by wearable device 500.

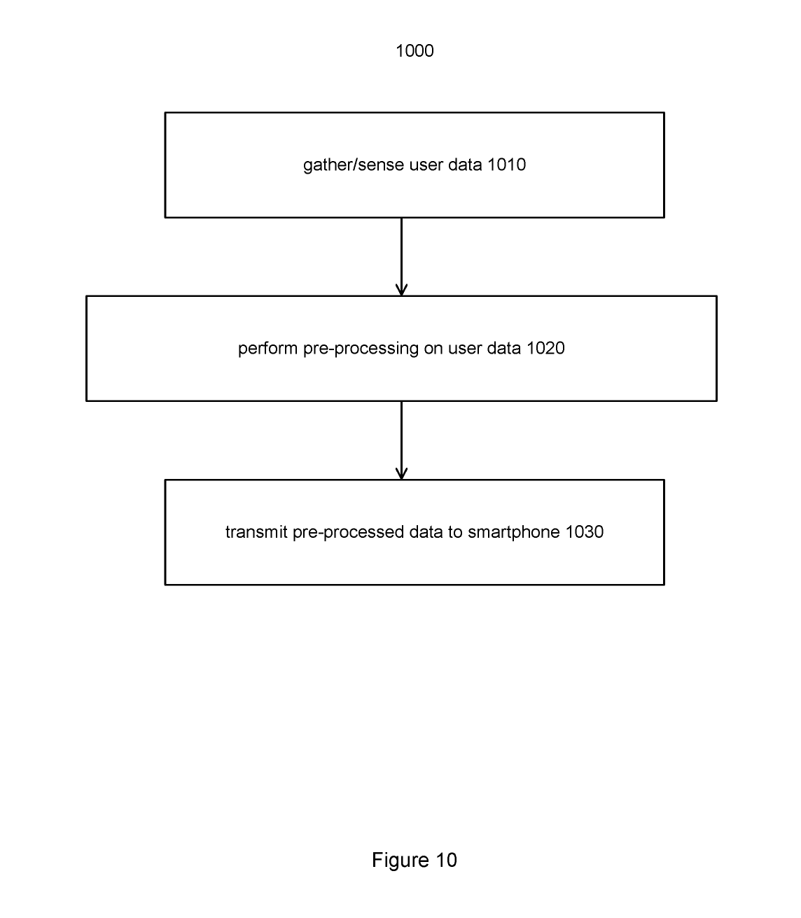

FIG. 10 is a data capture process 1000 that may be performed by wearable device 500 according to an embodiment of the invention. In 1010, wearable device 500 may gather data that can be processed to provide an indication of the wearer's BP as discussed above. In 1020, wearable device 500 may perform pre-processing, such as amplification and/or filtration of the gathered data, as discussed above. In some embodiments, pre-processing may be omitted or may be optional. In 1030, wearable device 500 may transmit the pre-processed data to smartphone 910 via network 900. Wearable device 500 may perform process 1000 periodically and/or substantially continuously (e.g., repeated once per second, once per minute, or in other frequent intervals). In some embodiments, wearable device 500 may perform the gathering and/or pre-processing periodically and/or continuously and send pre-processed data to smartphone 910 in batches.

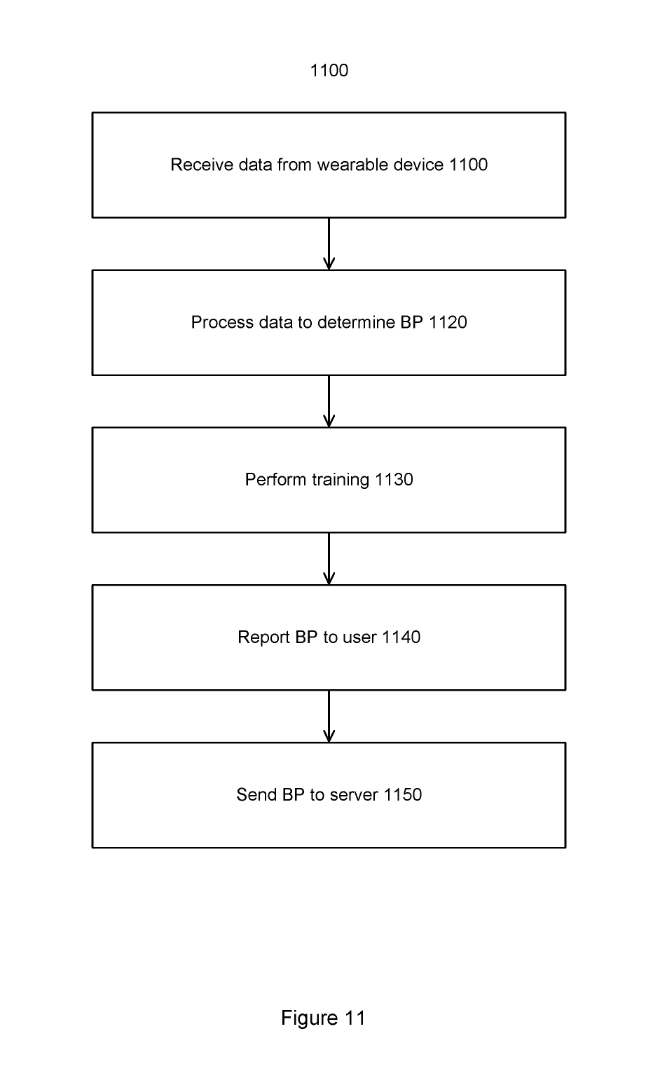

FIG. 11 is a data analysis process 1100 according to an embodiment of the invention. In 1110, smartphone 910 may receive pre-processed data from wearable device 500. In 1120, smartphone 910 may process the pre-processed data to determine BP. For example, smartphone 910 may apply one or more of the procedures disclosed above in the “Blood Pressure Measurement” section of this document.

In 1130, during an initial use of wearable device 500 and/or smartphone 910, smartphone 910 may perform training using the pre-processed data and generated BP. For example, the BP of user may be measured 5 times in order to train the BP derivation algorithm. Personal constants may be found, associated with a personal ID, and saved. Constants may be saved in smartphone 910 memory and/or in the cloud (see 1150). Optionally, manually simulated ECG and BP may be used to test the program to investigate how “smart” it is, and the algorithm may be adjusted accordingly. In subsequent uses of wearable device 500 and/or smartphone 910, this training may be omitted.

In 1140, smartphone 910 may display BP on its display to inform the user of his or her current BP. BP may be updated continuously and displayed in response to a user command, for example.

In 1150, smartphone 910 may send BP data (including constants after a training) to a remote server. With the application, the user may choose to send the data to a specific registered doctor or store for further diagnoses. The application itself may include an algorithm to judge the data and choose only a valuable or relevant subset of data to send and/or store (e.g., most recent data, data not clearly an outlier, data indicating a change in condition, etc.).

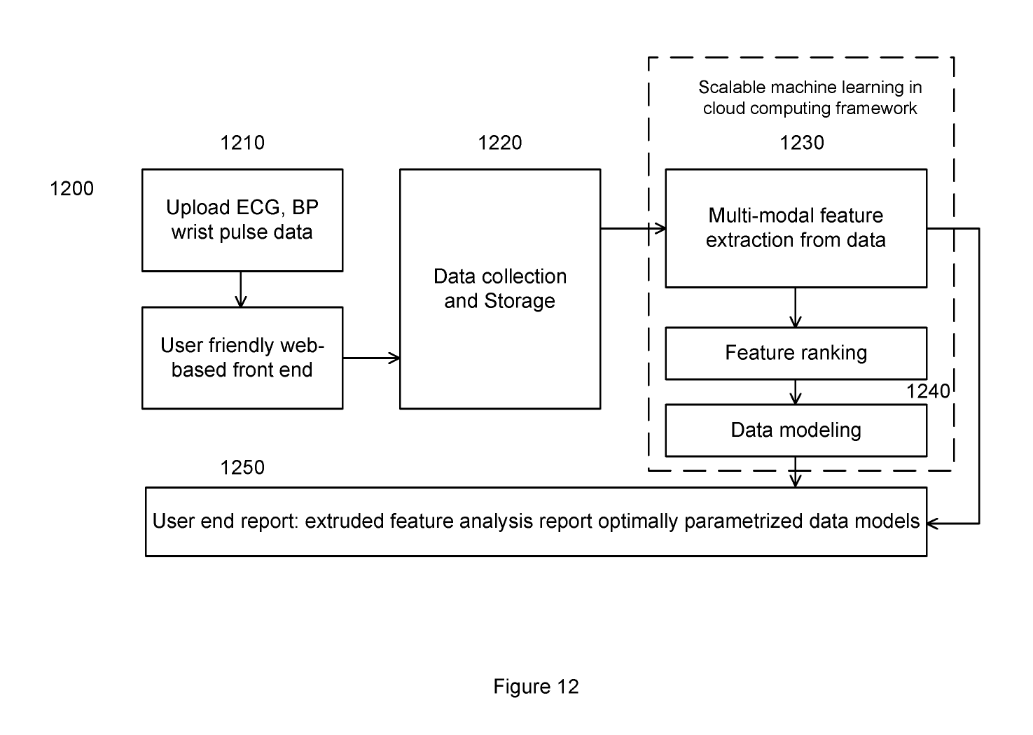

FIG. 12 is a data collection process 1200 according to an embodiment of the invention. Data collection process 1200 may be performed by one or more servers (e.g., a “cloud computing platform”) with which smartphone 910 is configured to communicate (e.g., through the Internet or another network).

In 1210, the platform may receive data uploaded by smartphone 910. In 1220, the platform may store the data. The cloud computing platform may leverage a hardware independent scalable framework that can store a variety of data sizes.

In 1230, the platform may identify multi-modal features from the data sets per user. These features may include statistical parameters extracted from the temporal data. A most discriminating set of features may be identified by scalable feature ranking methods such as F-score, AdaBoost, and minimum Redundancy Maximum Relevance (mRMR). Using the reduced set of most discriminating features, several classification and regression data models may be parameterized, such as support vector machines (SVM), AdaBoost, Gaussian Mixture models, logistic regression, etc. Each data model may be optimally parameterized using a training data set to minimize the data modeling errors on the validation data sets.

In 1240, the data model with overall minimum validation errors across all data models may be selected for BP data modeling and outlier reporting on the test data sets. Suitable user-end reports may be generated for each of the computing phases.

The scalable machine learning framework may be designed to invoke hyper-parameterization across multiple data models and select the optimal data model per patient. This process may ensure the robustness and reliability of the data modeling process. In 1250, the platform may return the model to smartphone 910 through the network. The smartphone application may take this individualized model and calculate, visualize, communicate, and store the BP information. This may allow the application to run accurately without the first-time calibration in a situation such as the user changing phones. Periodic re-calibration may be performed if the user undergoes physiological changes, like weight loss or gain, or upon the incidence of other medical conditions that might affect the BP computation, for example.

As described above, the systems and methods disclosed herein may provide an easy to use and hassle free wearable system for continuously monitoring BP. Wearable devices may be configured to comfortably wrap around the upper arm and be hidden under clothes and/or may be integrated into wrist-worn devices such as watches or bracelets, for example. Stretchable circuits may allow these devices to fit a variety of different users. Low power circuit elements may ensure long battery life and low cost. Results provided and displayed to users and/or medical professionals may be simple and easy to interpret.

It should be appreciated that the examples set forth above are provided merely for the purpose of explanation and are in no way to be construed as limiting. While reference to various embodiments is made, the words used herein are words of description and illustration, rather than words of limitation. Further, although reference to particular means, materials, and embodiments are shown, there is no limitation to the particulars disclosed herein. Rather, the embodiments extend to all functionally equivalent structures, methods, and uses, such as are within the scope of the appended claims.

What is claimed is:

1. A wireless monitoring method comprising:
detecting, by at least one sensor in a wearable device worn by a user, at least one of an electrocardiogram (ECG) signal and arterial pulse or photoplethysmogram (PPG) signal;
processing, by at least one processing circuit, the ECG signal, the arterial pulse or PPG signal, or both to determine at least one of a pulse transit time (PTT) and pulse arrival time (PAT);
determining, by the at least one processing circuit, a blood pressure (BP) of the user from the PTT, the PAT, or both.

2. The wireless monitoring method of claim 1, wherein the at least one sensor detects the ECG signal and/or arterial pulse or PPG signal from only one side of a body of the user.

3. The wireless monitoring method of claim 1, wherein the detecting is performed at a substantially continuous rate.

4. The wireless monitoring method of claim 1, wherein the BP is determined at the substantially continuous rate.

5. The wireless monitoring method of claim 1, further comprising reporting, by the at least one processing circuit, the BP to the user.

6. The wireless monitoring method of claim 1, further comprising transmitting, by at least one communication circuit coupled to the at least one processing circuit, the BP to a server.

7. The wireless monitoring method of claim 1, further comprising calibrating, by the at least one processing circuit, the at least one processing circuit to determine the BP based on at least one user characteristic, at least one sensor characteristic, or a combination thereof.

8. The wireless monitoring method of claim 1, further comprising:
filtering, by a filtering circuit, the ECG signal, the arterial pulse or PPG signal, or both prior to the processing; or
amplifying, by an amplifying circuit, the ECG signal, the arterial pulse or PPG signal, or both prior to the processing; or
a combination thereof.

9. A wearable device comprising:
a flexible band configured to be worn by a user; and
a flexible circuit coupled to the flexible band, the flexible circuit comprising:
at least one sensor configured to detect at least one of an electrocardiogram (ECG) signal and arterial pulse or photoplethysmogram (PPG) signal;
a processing circuit configured to generate pre-processed data by performing at least a portion of processing the ECG signal, the arterial pulse or PPG signal, or both to determine at least one of a pulse transit time (PTT) and pulse arrival time (PAT) and determining a blood pressure (BP) of the user from the PTT, the PAT, or both; and
a communication circuit configured to transmit the pre-processed data.

10. The wearable device of claim 9, wherein the at least one sensor is coupled to the flexible band such that the at least one sensor is positioned on only one side of a body of the user when the flexible band is worn by the user.

11. The wearable device of claim 9, wherein:
the at least one sensor is configured to detect the ECG signal, the arterial pulse or PPG signal, or both at a substantially continuous rate; and
the processing circuit is configured to generate the pre-processed data at the substantially continuous rate.

12. The wearable device of claim 9, further comprising:
a filtering circuit between the at least one sensor and the processing circuit configured to filter the ECG signal, the arterial pulse or PPG signal, or both prior to the processing; or
an amplifying circuit between the at least one sensor and the processing circuit configured to amplify the ECG signal, the arterial pulse or PPG signal, or both prior to the processing; or
a combination thereof.

13. The wearable device of claim 9, wherein the at least one sensor comprises at least one electrode configured to protrude through the flexible band.

14. The wearable device of claim 13, wherein a protruding portion of each at least one electrode configured to protrude through the flexible band is formed by electroplating.

15. The wearable device of claim 9, wherein the at least one sensor comprises at least one pulse pressure sensor, at least one non-contact electrode, or a combination thereof.

16. The wearable device of claim 9, wherein the flexible circuit further comprises a power supply.

17. A system comprising:
the wearable device of claim 9; and
a computing device comprising:
a communication circuit configured to receive the pre-processed data from the wearable device; and
a processing circuit configured to further process the pre-processed data by performing at least a portion of processing the ECG signal, the arterial pulse or PPG signal, or both to determine at least one of a pulse transit time (PTT) and pulse arrival time (PAT) and determining a blood pressure (BP) of the user from the PTT, the PAT, or both.

18. The system of claim 17, wherein the computing device further comprises a display configured to display the BP.

19. The system of claim 17, wherein the computing device is further configured to communicate, by the communication circuit, the BP to a remote server.

20. The system of claim 17, wherein the computing device is further configured to receive, by the communication circuit, data related to the BP from a remote server.
